(19) United States
(12) Patent Application Publication
(10) Pub. No.: US 2020/0185078 A1
(43) Pub. Date: Jun. 11, 2020

(54) SYSTEM AND METHOD FOR FACILITATING CONFIGURATION MODIFICATIONS FOR A PATIENT INTERFACE COMPUTER SYSTEM BASED ON A PATIENT-SPECIFIC RISK ALERT MODEL

(71) Applicant: KONINKLIJKE PHILIPS N.V., EINDHOVEN (NL)

(72) Inventors: Steffen Clarence PAUWS, Eindhoven (NL); Daniele DE MASSARI, Eindhoven (NL); Nicolaas Gregorius Petrus DEN TEULING, Eindhoven (NL); Ioanna SOKORELI, Eindhoven (NL); Jarno Mikael RIISTAMA, Waalre (NL)

(21) Appl. No.: 16/608,445

(22) PCT Filed: Apr. 24, 2018
(86) PCT No.: PCT/EP2018/060461

§ 371 Date: Oct. 25, 2019

Related U.S. Application Data
(60) Provisional application No. 62/489,697, filed on Apr. 25, 2017.

(51) Int. Cl. G16H 20/13; G16H 50/30; G16H 40/67; A61B 5/00

(52) U.S. Cl. CPC G16H 20/13; A61B 5/0022; G16H 40/67; G16H 50/30

(57) ABSTRACT

The present disclosure pertains to a system for facilitating configuration modifications for a patient interface computer system based on a patient-specific risk alert model. In some embodiments, the system obtains (i) lifestyle information associated with a patient, (ii) disease information associated with the patient, and (iii) one or more physiological measurements of the patient. The system monitors the patient for one or more threshold levels of exacerbation based on a risk alert model, the lifestyle information, the disease information, and the one or more physiological measurements. The system causes a configuration of the patient interface computer system to be modified based on the monitoring for the one or more threshold levels of exacerbation.

### BACKGROUND

### 1. Field

The present disclosure pertains to a system and method for facilitating a patient interface computer system and configuration modifications for the patient interface computer system based on a patient-specific risk alert model.

### 2. Description of the Related Art

Chronically ill patients are managed by telehealth services to prevent hospital readmissions for an emergency and to improve the patients' quality of life. Although computer-assisted readmission risk determination systems exist, such risk determination systems fail to consider many disease and patient related factors that may lead to variation in prognosis. Thus, such risk determination systems may be agnostic to the precise diagnosis, etiology, co-morbidities, post-discharge patient condition, prevailing social support, patient frailty, patient engagement, or other factors which may lead to inadequate patient follow-up and decision making with respect to deterioration trajectories.

### SUMMARY

Accordingly, one or more aspects of the present disclosure relate to a system configured to facilitate configuration modifications for a patient interface computer system based on a patient-specific risk alert model. The system comprises one or more processors or other components. The one or more processors are configured by machine-readable instructions to: obtain lifestyle information associated with a patient; obtain disease information associated with the patient; obtain one or more physiological measurements of the patient via one or more sensors, the one or more sensors being configured to provide real-time signals conveying information indicating the one or more physiological measurements of the patient; monitor the patient for one or more threshold levels of exacerbation based on a risk alert model, the lifestyle information, the disease information, and the one or more physiological measurements; and cause a configuration of the patient interface computer system to be modified based on the monitoring for the one or more threshold levels of exacerbation.

Yet another aspect of the present disclosure relates to a method for facilitating configuration modifications for a patient interface computer system based on a patient-specific risk alert model with a system. The system comprises one or more processors or other components. The method comprises: obtaining, with the one or more processors, lifestyle information associated with a patient; obtaining, with the one or more processors, disease information associated with the patient; obtaining, via one or more sensors, one or more physiological measurements of the patient, the one or more sensors being configured to provide real-time signals conveying information indicating the one or more physiological measurements of the patient; monitoring, with the one or more processors, the patient for one or more threshold levels of exacerbation based on a risk alert model, the lifestyle information, the disease information, and the one or more physiological measurements; and causing, with the one or more processors, a configuration of the patient interface computer system to be modified based on the monitoring for the one or more threshold levels of exacerbation.

Still another aspect of present disclosure relates to a system for facilitating configuration modifications for a patient interface computer system based on a patient-specific risk alert model. The system comprises: means for obtaining lifestyle information associated with a patient; means for obtaining disease information associated with the patient; means for obtaining one or more physiological measurements of the patient, the means for obtaining one or more physiological measurements being configured to provide real-time signals conveying information indicating the one or more physiological measurements of the patient; means for monitoring the patient for one or more threshold levels of exacerbation based on a risk alert model, the lifestyle information, the disease information, and the one or more physiological measurements; and means for causing a configuration of the patient interface computer system to be modified based on the monitoring for the one or more threshold levels of exacerbation.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system for facilitating a patient interface computer system, in accordance with one or more embodiments.

FIG. 2 illustrates a medication dispenser, in accordance with one or more embodiments.

FIG. 3 illustrates a method for facilitating configuration modifications for a patient interface computer system based on a patient-specific risk alert model, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

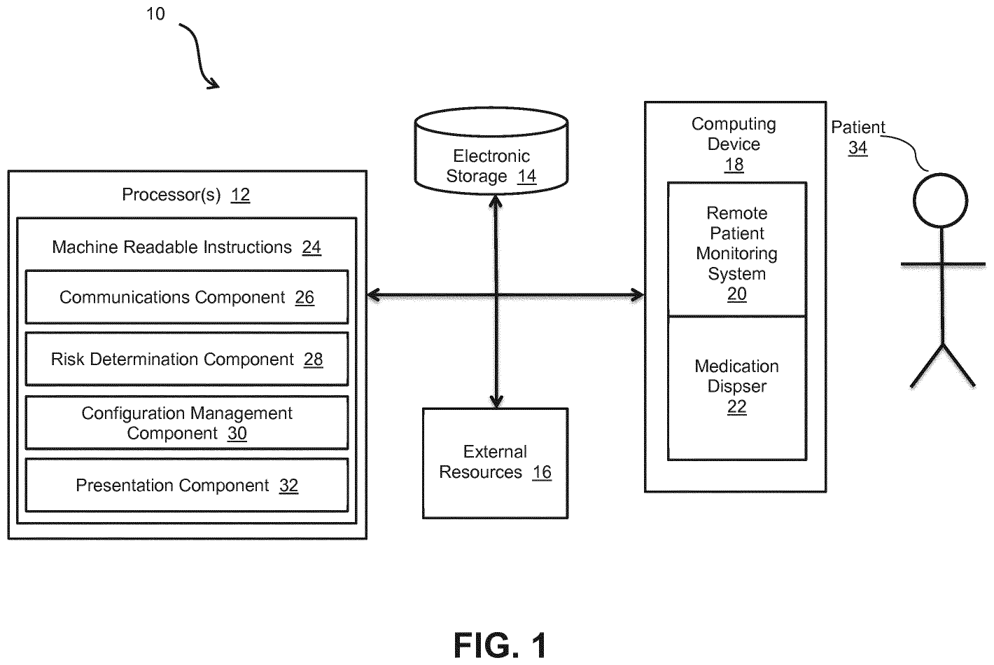

FIG. 1 is a schematic illustration of a system 10 for facilitating a patient interface computer system. In some embodiments, system 10 is configured to determine probabilities of deterioration based on a baseline patient status. In some embodiments, system 10 is configured to obtain lifestyle information, disease information, and one or more physiological measurements associated with a patient to monitor the patient for one or more threshold levels of exacerbation. In some embodiments, system 10 is configured to automatically select a telehealth program for the patient based on the determined baseline patient status. In some embodiments, system 10 is configured to determine an impact of one or more telehealth program by retrospectively analyzing outcomes of one or more prior telehealth programs in relation to previously determined baseline probabilities. In some embodiments, system 10 is configured to determine one or more rankings for one or more telehealth programs and/or interventions based on their expected impact. In some embodiments, system 10 is configured to recommend a telehealth program and/or an intervention having the highest ranking and/or other rankings to a patient. In some embodiments, system 10 is configured to determine the baseline probability and monitor the patient for one or more threshold levels of exacerbation using a logistic regression model.

In some embodiments, system 10 is configured to determine baseline patient status at telehealth enrollment to improve clinical deterioration detection during telehealth service and cause a configuration of a patient interface computer system to be modified based on monitoring for one or more threshold levels of exacerbation (e.g., adjust or recommend change to an amount of medication dispensed, frequency of medication dispensing, an amount of monitoring of the patient with a remote patient monitoring system, etc.). In some embodiments, system 10 comprises one or more processors 12, electronic storage 14, external resources 16, computing device 18, or other components.

In some embodiments, computing device 18 includes a remote patient monitoring system 20 and a medication dispenser 22. Remote patient monitoring system may include one or more sensors configured to provide real-time signals conveying information indicating one or more physiological measurements, disease information associated with patient 34, or other information. In some embodiments, the sensors include (i) equipment used in hospitals, doctor's offices, or other medical facilities, in the home of patient 34, or in other locations to monitor vital signs or other physiological information (e.g., pulse rate monitors, blood pressure monitors, blood oxygenation monitors, glucose monitors, weight scales, thermometers, electrocardiogram (EKG) equipment, childbirth labor contraction monitors, etc.), (ii) test equipment (e.g., imaging equipment such as an MRI or an x-ray machine, an ultrasound, electroencephalogram (EEG) equipment, etc.), (iii) equipment for treating patient 34 (e.g., respirators/ventilators, light therapy devices, etc.), or (iv) other sensors. In some embodiments, remote patient monitoring system 20 includes one or more wearable devices including the sensors (e.g., Apple Watch, Fitbit, Philips Health Watch, etc.). In some embodiments, information from the sensors may be automatically transmitted to computing device 18, one or more remote servers, or other destinations via one or more networks (e.g., local area networks, wide area networks, the Internet, etc.) on a periodic basis, in accordance to a schedule, or in response to other triggers.

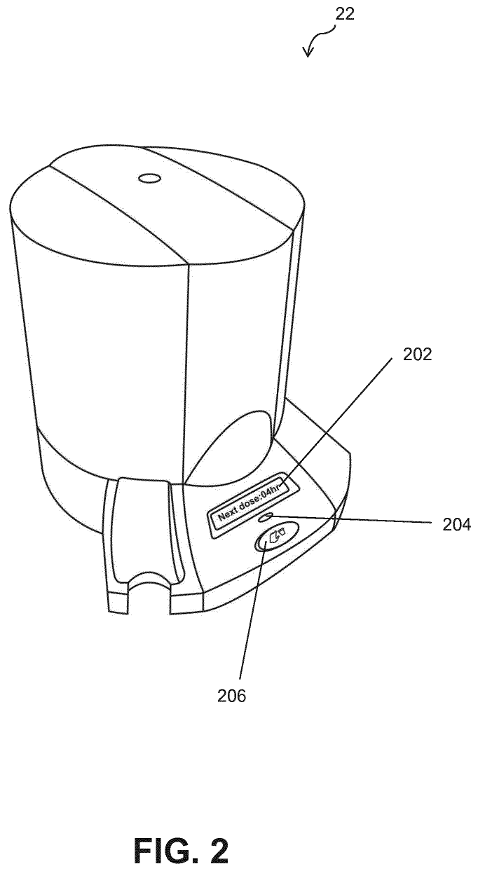

By way of a non-limiting example, FIG. 2 illustrates a medication dispenser, in accordance with one or more embodiments. As shown in FIG. 2, medication dispenser 22 includes a display 202, notification module 204, dispensing button 206, or other components. In some embodiments, medication dispenser 22 is configured to present, via display 202, a time remaining to a next medication dose. In some embodiments, medication dispenser 22 is configured to present, via display 202, medication information associated with dispensed medication or medication included in a next dose. In some embodiments, medication information includes medication name, medication dosage, prescribing physician, allergen information, or other information. In some embodiments, medication dispenser 22 is configured to notify, via notification module 204, patient 34 of a scheduled medication consumption. For example, notification module includes one or more of an indicator light, an audible alarm, a tactile engine, or other modules.

In some embodiments, medication dispenser 22 is configured to automatically dispense medication on a periodic basis, in accordance with a schedule, or in response to other triggers. In some embodiments, medication dispenser 22 is configured to automatically dispense in a prescribed amount, an amount determined by configuration management component 30 (e.g., as described below), or other amounts approved by one or more caregivers at a scheduled time (e.g., on a periodic basis, in accordance with a schedule, or in response to other triggers approved by one or more caregivers and/or as determined by configured management component 30 (e.g., as described below)).

In some embodiments, medication dispenser 22 is configured to provide a report associated with patient 34's adherence with a care plan, prescription, or other medical guidelines. As an example, medication dispenser 22 may automatically transmit the report to one or more caregivers via a network on a periodic basis, in accordance to a schedule, or in response to other triggers. In some embodiments, medication dispenser 22 is configured to notify one or more caregivers regarding one or more missed medication doses. As an example, medication dispenser 22 may notify the one or more caregivers responsive to dispensing button 206 not being pressed within a predetermined amount of time of a scheduled medication consumption (e.g., as indicated by notification module 204).

Computing device 18 is configured to provide an interface between patient 34, caregivers, and system 10. In some embodiments, computing device 18 is associated with individual caregivers, a central caregiver coordinator, or other users. Computing device 18 is configured to provide information to or receive information from patient 34, caregivers, or other users. Computing device 18 includes a user interface or other components. The user interface may be or include a graphical user interface configured to present caregivers with views or fields configured to receive entry or selection of information related to patient 34 or provide/receive other information. In some embodiments, the user interface includes a plurality of separate interfaces associated with a plurality of computing devices 18, processor 12, or other components of system 10.

In some embodiments, computing device 18 is configured to provide the user interface, processing capabilities, databases, or electronic storage to system 10. As such, computing device 18 may include processor 12, electronic storage 14, external resources 16, or other components of system 10. In some embodiments, computing device 18 is connected to a network (e.g., the internet). In some embodiments, computing device 18 does not include processor 12, electronic storage 14, external resources 16, or other components of system 10, but instead communicate with these components via the network. The connection to the network may be wireless or wired. For example, processor 12 may be located in a remote server and may wirelessly cause presentation of the risk of readmission via the user interface to a caregiver on a computing device 18 associated with that caregiver (e.g., a doctor, a nurse, a central caregiver coordinator, etc.). In some embodiments, computing devices 18 are laptops, desktop computers, smartphones, tablet computers, or other computing devices.

Examples of interface devices suitable for inclusion in the user interface include a camera, a touch screen, a keypad, touch sensitive or physical buttons, switches, a keyboard, knobs, levers, a display, speakers, a microphone, an indicator light, an audible alarm, a printer, tactile haptic feedback device, or other interface devices. The present disclosure also contemplates that computing device 18 includes a removable storage interface. In this example, information may be loaded into computing device 18 from removable storage (e.g., a smart card, a flash drive, a removable disk, etc.) that enables caregivers or other users to customize the implementation of computing device 18. Other exemplary input devices and techniques adapted for use with computing device 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device 18, devices that are part of external resources 16, electronic storage 14, or other devices.)

In some embodiments, processor 12, electronic storage 14, external resources 16, computing device 18, or other components may be operatively linked via one or more electronic communication links. For example, such electronic communication links may be established, at least in part, via a network such as the Internet, or other networks. It will be appreciated that this is not intended to be limiting, and that the scope of this disclosure includes embodiments in which these components may be operatively linked via some other communication media. In some embodiments, processor 12 is configured to communicate with electronic storage 14, external resources 16, computing device 18, or other components according to a client/server architecture, a peer-to-peer architecture, or other architectures.

As shown in FIG. 1, processor 12 is configured via machine-readable instructions 24 to execute one or more computer program components. The computer program components may comprise one or more of a communications component 26, a risk determination component 28, a configuration management component 30, a presentation component 32, or other components. Processor 12 may be configured to execute components 26, 28, 30, or 32 by software; hardware; firmware; some combination of software, hardware, or firmware; or other mechanisms for configuring processing capabilities on processor 12.

It should be appreciated that although components 26, 28, 30, and 32 are illustrated in FIG. 1 as being co-located within a single processing unit, in embodiments in which processor 12 comprises multiple processing units, one or more of components 26, 28, 30, or 32 may be located remotely from the other components. The description of the functionality provided by the different components 26, 28, 30, or 32 described below is for illustrative purposes, and is not intended to be limiting, as any of components 26, 28, 30, or 32 may provide more or less functionality than is described. For example, one or more of components 26, 28, 30, or 32 may be eliminated, and some or all of its functionality may be provided by other components 26, 28, 30, or 32. As another example, processor 12 may be configured to execute one or more additional components that may perform some or all of the functionality attributed below to one of components 26, 28, 30, or 32.

Communications component 26 is configured to obtain lifestyle information associated with patient 34. In some embodiments, the lifestyle information includes demographics information associated with patient 34, patient engagement/activation information, social support information, frailty assessment information, socio-economic status information, information indicating medical-psychological constructs (e.g., disease-related depression, anxiety, etc.), or other information. In some embodiments, patient demographics information includes one or more of an age, a gender, years of education, whether or not patient 34 lives alone, marital status, number of children, or other information. In some embodiments, the social-support information may indicate a parameter associated with a level of support from one or more informal care givers associated with the patient (e.g., partner/spouse, family members/relatives, friends/peers, neighbors, etc.). The activation information may indicate a parameter associated with a score value indicating a level of patient engagement to a treatment (e.g., a number of missed medication doses, etc.). The frailty information may indicate a parameter associated with a score indicating frailty. For example, frailty is present if a patient has three or more of the following criteria: unintentional weight loss (≥10 lbs in the past year), self-reported exhaustion, weakness (reduced grip, reduced strength for sex, and reduced body mass index (BMI)), slow walking speed, low physical activity, and/or other criteria). In some embodiments, communications component 26 is configured to identify patient 34 as prefrail in response to one or two of the frailty criteria being present. In some embodiments, communications component 26 is configured to identify patient 34 as frail in response to three or more of the frailty criteria being present. In some embodiments, patient 34 is asked to respond to a question about having trouble bathing or dressing and/or performing ‘get up and go’ test. In some embodiments, the ‘timed get up and go’ test requires patient 34 to stand up from a chair, walk a short distance (e.g., 3 meters), turn around, return, and sit down again. In some embodiments, a normal time to complete the ‘get up and go’ test is less than 10 seconds. In some embodiments, responsive to patient 34 (i) reporting having trouble in bathing or dressing and/or (ii) completing the ‘get up and go’ test in more than 20 seconds, communications component 26 is configured to identify patient 34 as frail. The severity information may indicate a parameter associated with a level of disease severity (e.g., NT-proBNP, NYHA, etc.). In some embodiments, lifestyle information include manually recorded information, results of an electronic survey, information in medical care provider databases (e.g., Medicare databases), or other information. In some embodiments, communications component 26 is configured to facilitate direct entry or selection of lifestyle information by patient 34 via computing device 18. In some embodiments, communications component 26 obtains lifestyle information stored in electronic storage 14, information stored in electronic medical record systems or other medical information systems of care providers associated with system 10 (e.g., servers or other databases that are part of external resources 16 such as Medicare databases, etc.), or information from other sources.

In some embodiments, communications component 26 is configured to obtain disease information associated with patient 34. In some embodiments, disease information includes one or more of an etiology, a diagnosis, a co-morbid, a post-discharge, clinical markers, medication prescription, disease syndromes/symptoms, or other information. In some embodiments, the etiology indicates a parameter associated with ischaemic or non-ischaemic heart failure (e.g., ischaemic due to Myocardial infarction, non-ischaemic due to hypertension), the diagnosis indicates a parameter associated with systolic vs diastolic blood pressure, and left vs right heart failure, the co-morbid indicates a parameter associated with one or more co-morbidities of the patient (e.g., COPD, Diabetes Mellitus, aneamie, renal dysfunction, etc.), the post-discharge indicates a parameter associated with one or more patient conditions at discharge (e.g., dry weight status, normal jugular venous pressure, malnutrition), the clinical markers indicate a disease severity (e.g., LVEF, NT-proBNP, etc.), the medication prescription includes one or more of ACEi/ARB/Beta/Diuretics doses, and the disease syndromes/symptoms include cachexia and pulmonary vs peripheral oedema. In some embodiments, the disease information includes manually recorded information, test results, output from remote patient monitoring system 20 or other medical devices, information in medical care provider databases (e.g., Medicare databases) or other information. In some embodiments, communications component 26 obtains disease information associated with patient 34 stored in electronic storage 14, information stored in electronic medical record systems or other medical information systems of care providers associated with system 10 (e.g., servers or other databases that are part of external resources 16 such as Medicare databases, etc.), or information from other sources.

In some embodiments, communications component 26 is configured to obtain one or more physiological measurements of the patient via remote patient monitoring system 20.

Risk determination component 28 is configured to monitor the patient for one or more threshold levels of exacerbation based on a risk alert model, the lifestyle information, the disease information, the physiological measurements, or other information. In some embodiments, risk determination component 28 is configured such that monitoring the patient for the one or more threshold levels of exacerbation comprises (i) determining a baseline probability with a logistic regression model based on the lifestyle information and the disease information and (ii) determining a probability of exacerbation with the risk alert model based on the determined baseline probability and the one or more physiological measurements. In some embodiments, risk determination component 28 may be configured to determine the covariates to be used in a logistic regression model based on information availability. Logistic regression is a statistical technique used to predict a binary prognostic outcome, such as death, morbidity, hospital (re-)admission, an exacerbation, and/or other outcomes. In some embodiments, responsive to a determination of missing information (e.g., 15% of the patients lack data for a co-variate), risk determination component 28 may be configured to substitute missing information elements with estimated information determined via one or more data imputation methods. As an example, missing information is determined by data lacking any entry or undefined value. In some embodiments, missing information is caused by an administrative error, an incident during a measurement or data collection session, unavailability of patient 34 due to sickness, patient 34 being unable/unavailable to perform a test for which data needs to be collected, a local health facility (e.g., local protocol or organization) not collecting a particular data or performing a particular (diagnostic) test, and/or other factors.

In some embodiments, risk determination component 28 is configured to determine a baseline probability by determining a probability of exacerbation based on the lifestyle information and the disease information. In some embodiments, risk determination component 28 is configured to determine the baseline probability on a continuous, daily, weekly, or other periodic basis. By way of a non-limiting example, Equation 1 describes the binary outcome of an exacerbation within a time period (e.g., 30 days), denoted as “Z=1”, which is linked to a linear combination of predictors, denoted as “Xᵢ”, that are weighted by (regression) coefficients βᵢ.

Xβ=β₀+β₁X₁+ . . . +βₚXₚ. (1)

As shown in Equation 1, the baseline predictors denoted as X include one or more of age, gender, etiology, diagnosis, co-morbid, post-discharge, and or other factors. In some embodiments, the baseline predictors denoted as X include one or more of age, gender, social-support, activation, frailty, severity, and/or other factors.

In some embodiments, the probability of the occurrence of the outcome of Equation 1 is modeled by a logistic function to determine a baseline probability. By way of a non-limiting example, Model 1 describes a probability of exacerbation as a baseline probability.

Model 1: P(Z=1|X)=(1+exp(−Xβ))⁻¹. As shown in Model 1, the coefficients βᵢ are estimated by a maximum likelihood method.

In some embodiments, risk determination component 28 is configured to determine the baseline probability based on a local patient cohort (e.g., a locally prevailing statistically related group of patients that are (i) treated by local care providers, (ii) in a set of specialties or units, (iii) from a local socio-economic environment) by recalibrating the logistic model. In some embodiments, risk determination component 28 is configured such that the recalibration includes changing the intercept and the slope of the model (i.e., the βᵢ coefficients, where i=0, 1, . . . , p) by using locally collected information.

By way of a non-limiting example, the linear predictor Xβ=β₀+β₁X₁+ . . . +βₚXₚ, as provided in Equation 1, may be calibrated based on new data by fitting the linear predictor in Model 2.

Model 2: logit (Z)=a+b×Xβ. In Model 2 a maximum likelihood estimation may be incorporated to determine the recalibration intercept ‘a’ and the calibration slope ‘b’.

In some embodiments, risk determination component 28 is configured to re-calibrate the baseline probability during a planned outpatient visit, after a medical event, or after a defined period on telehealth.

In some embodiments, risk determination component 28 is configured to monitor patient 34 for one or more threshold levels of exacerbation with a risk alert model based on the determined baseline probability and the physiological measurements (e.g., daily vital sign readings). In some embodiments, risk determination component 28 is configured such that the risk alert model determines a probability of an exacerbation for patient 34 on a continuous (e.g., without a predetermined frequency and/or responsive to new data points being collected), daily, weekly, or other periodic basis. In some embodiments, the risk alert model includes determining a probability of an exacerbation via a logistic regression model. In some embodiments, the binary outcome of an exacerbation within a time period, denoted as “Z=1”, is linked to a linear combination of predictors, denoted as “Yᵢ”, that are weighted by (regression) coefficients γᵢ. By way of a non-limiting example, equation 2 describes the binary outcome of an exacerbation.

Yγ=γ₀γ₁Y₁+ . . . +γₗYₗ+γₗ₊₁P(Z=1|X). (2)

As shown in Equation 2, the predictors denoted as Y include one or more of body weight, heart rate, blood pressure, shortness of breath, fatigue, swelling of ankles, and/or other factors. P(Z=1|X) denote the baseline probability value.

In some embodiments, the probability of the occurrence of the outcome of Equation 2 is modeled by a logistic function. By way of a non-limiting example, Model 3 describes a risk alert model (e.g., a logistic regression model) for determining a probability of an exacerbation for patient 34 based on the determined baseline probability and the physiological measurements.

Model 3: P(Z=1|Y, baseline)=(1+exp(−Yγ))⁻¹. As shown in Model 3, the baseline probability value as determined by Model 1 is put into the model as a co-variate.

In some embodiments, risk determination component 28 is configured to monitor patient 34 for one or more threshold levels of exacerbation based on a probability of exacerbation determined at baseline via Bayesian reasoning. In some embodiments, risk determination component 28 is configured to determine a posterior probability from a prior probability of exacerbation determined at baseline and a likelihood function of observing telehealth data on daily measurements and self-reports given exacerbation has taken place. In some embodiments, risk determination component 28 is configured to omit the determination of the probability of observing a given daily measurement to obtain a likelihood estimate that is proportional to the probability of exacerbation. By way of a non-limiting example, Model 4 describes Bayesian reasoning used to update the probability of an exacerbation.

Model 4: P(Z=1|physiological measurement)∝P(physiological measurement|Z=1)×P(Z=1|X). As shown in Model 4, the posterior probability of P(Z=1|physiological measurement) is proportional to the a priori baseline (e.g., as determined by Model 1) P(Z=1|X) and the likelihood P (physiological measurements|Z=1). The likelihood probability indicates a probability of observing a particular value for a physiological measurement such as a body weight value, a heart rate value or a blood pressure value under an exacerbation (or admission), that is, the hypothesis Z=1. In some embodiments, risk determination component 28 is configured to determine a number of times in which a particular physiological measurement is observed while (i) patient 34 is exacerbating and (ii) patient 34 is not exacerbating to obtain the likelihood. In some embodiments, the baseline is proportional as it needs to be normalized by a marginal likelihood.

For example, a number of observations for an increase of more than 2 kilograms in body weight in patient 34 over the last two days may be 0.75 in case of an exacerbation (Z=1) and 0.4 in case of no exacerbation (Z=0). As such, P(physiological measurement|Z=1)=0.75 and P(physiological measurement|Z=0)=0.4. In this example, it may be assumed that the baseline P(Z=1|X)=0.2. Based on the Bayes reasoning the posterior probability of an exacerbation is proportional to the product of P(physiological measurement|Y=1) and the priori baseline. In this example, the probabililty is determined as (0.75×0.2)/(0.75×0.2+0.4×0.8)=0.32, wherein the denominator term (0.75×0.2+0.4×0.8) denotes the marginal likelihood. As described above, with the new observation of an increase in the body weight the risk estimate for an exacerbation has been increased from 0.2 at baseline to 0.32 a posteriori.

In some embodiments, risk determination component 28 is configured to stratify patient 34 or other patients into one or more threshold levels of exacerbation. For example, risk determination component 28 is configured to categorize patient 34 or other patients as low baseline risk (e.g., up to 5% probability at baseline), medium baseline risk (e.g., from 5 to 20% probability at baseline) and high baseline risk (higher than 20% probability at baseline).

In some embodiments, the baseline probability indicates a probability of exacerbation for a first time period and wherein the risk alert model indicates a probability of exacerbation for a second time period. In some embodiments, the first time period is greater than the second time period. For example, the baseline probability may indicate a probability of exacerbation for a time period of 90 days (e.g., period in which the patient is expected to be on telehealth service) and the risk alert model may indicate a probability of exacerbation for a time period of one to five days (e.g., short term detection window for a patient on telehealth). In other examples, other time periods may be utilized for the first time period or the second time period.

In some embodiments, risk determination component 28 is configured to select a telehealth program for patient 34 based on the determined baseline probability. In some embodiments, risk determination component 28 is configured to determine an impact of one or more telehealth programs by retrospectively analyzing outcomes of the prior telehealth programs in relation to previously determined baseline probabilities. In some embodiments, risk determination component 28 is configured to (i) compare the determined baseline probability and the monitored threshold levels of exacerbation for patient 34 with a baseline probability and one or more monitored threshold levels of exacerbation associated with one or more other patients admitted to a different telehealth program and (ii) determine the most effective telehealth program based on the comparison, wherein the most effective telehealth program is results in the least (re-) hospitalizations due to baseline patient status during the time window in which telehealth is provided. In some embodiments, risk determination component 28 is configured to compare patient sub-groups with similar risk levels. In some embodiments, risk determination component 28 is configured to quantify an outcome in number of exacerbation requiring emergency admission across different baseline risk levels.

In some embodiments, risk determination component 28 is configured to identify one or more sub-groups of patients having similar baselines (e.g., group 1 having a baseline less than 5%, group 2 having a baseline between 5% and 20%, and group 3 having a baseline greater than 20%) prior to administering an intervention and/or deploying telehealth monitoring. In some embodiments, one or more patients within the same sub-group may be provided with different interventions. In some embodiments, risk determination component 28 is configured to compare baseline probabilities, obtained before and after completion of the interventions, corresponding to each of the one or more patients receiving different interventions to identify an intervention having the highest impact (e.g., having the highest pre-post intervention decrease in the baseline probability) per patient group.

Configuration management component 30 is configured to cause a configuration of a patient interface computer system to be modified based on the monitoring for the threshold levels of exacerbation. In some embodiments, the patient interface computer system may include one or more components of computing device 18, one or more wearable devices, or other computer systems. In some embodiments, configurations of the patient interface computer system to be modified may include one or more settings corresponding to remote patient monitoring system 20 (e.g., frequency of monitoring with one or more sensors, duration of monitoring with the sensors, a particular time of day designated for monitoring one or more vital signs, physiological parameters, or other information), dosage of medication administered to patient 34, or other configurations.

For example, the patient interface computer system may include remote patient monitoring system 20. In this example, configuration management component 30 may change an amount of monitoring of patient 34 with remote patient monitoring system 20. In some embodiments, remote patient monitoring may occur at a particular time of the day, repeatedly occur every hour, 4 hours, 8 hours, 12 hours, or other periods and frequencies. Responsive to patient 34 reaching higher threshold levels of exacerbation, configuration management component 30 may, for example, change monitoring frequency of remote patient monitoring system 20 from once daily to every hour. In some embodiments, responsive to patient 34 reaching lower threshold levels of exacerbation, configuration management component 30 may lower a monitoring frequency of remote patient monitoring system 20 from hourly to every eight hours.

As another example, the patient interface computer system may include medication dispenser 22. In this example, configuration management component 30 may adjust or recommend change to an amount of medication dispensed. In some embodiments, responsive to patient 34 reaching higher threshold levels of exacerbation, configuration management component 30 may, for example, adjust a medication dosage for patient 34 without user intervention. In this example, medication dispenser may be caused to dispense two diuretic pills daily instead of a prescribed dosage of one diuretic pill per day. In some embodiments, responsive to patient 34 reaching lower threshold levels of exacerbation, configuration management component 30 may prompt one or more caregivers to approve a proposed change to the medication dose. In some embodiments, responsive to patient 34 reaching one or more threshold levels of exacerbation, configuration management component 30 may cause medication dispenser 22 to present, via display 202, a recommended change to an amount of medication dispensed. In some embodiments, configuration management component 30 may adjust the periodic basis and/or the schedule for dispensing. In this example, responsive to patient 34 reaching higher threshold levels of exacerbation, configuration management component 30 may cause medication dispenser 22 to dispense one diuretic pill every 8 hours rather than once daily.

In some embodiments, configuration management component 30 may be configured to, responsive to patient 34 reaching one or more threshold levels of exacerbation, recommend introduction of a new drug or cessation of a prescribed drug.

In yet another example, the patient interface computer system may include a wearable device (e.g., a smart watch or other wearable device, etc.). Configurations of the wearable system to be modified may include notifications, one or more types of notifications (e.g., alarms, reminders, directions, etc.), a frequency of notifications (e.g., hourly, six times, four times, or twice daily, etc.), presenting or hiding one or more options from a user interface associated with the wearable device, or other configurations. In some embodiments, configuration management component 30 may be configured to, responsive to patient 34 reaching one or more threshold levels of exacerbation, cause the wearable device to provide (i) a notification to patient 34 regarding a change in diet, (ii) a reminder regarding taking a particular medication, (iii) directions to an appropriate care facility (e.g., emergency room), and/other instructions. In some embodiments, responsive to patient 34 reaching higher threshold levels of exacerbation, configuration management component 30 may increase a number of user interface options associated with the wearable device. For example, user interface options corresponding to requesting a remote consultation, a request for emergency care, directions to the closest emergency department, or other options may be presented. In some embodiments, responsive to patient 34 reaching lower threshold levels of exacerbation, configuration management component 30 may reduce, limit, or hide one or more user interface options associated with the wearable device.

In some embodiments, configuration management component 30 may be configured to, responsive to patient 34 reaching one or more threshold levels of exacerbation, notify one or more care givers to contact patient 34 via phone and/or other communication means. In some embodiments, configuration management component 30 may be configured to, responsive to patient 34 reaching one or more threshold levels of exacerbation, recommend enrollment in a coaching program, physical activity programs, and/or other programs to patient 34.

Presentation component 32 is configured to effectuate presentation of the determined baseline probability and the monitoring of patient 34 for one or more threshold levels of exacerbation to one or more care givers. In some embodiments, presentation component 32 is configured to notify the one or care givers regarding a change in the baseline probability for patient 34 or a change in the monitored threshold levels of exacerbation. For example, presentation component 32 may present an alert on a computing device (e.g., wearable device, laptop, desktop, etc.) user interface associated with the care givers indicating an increase in the baseline probability for patient 34. As another example, presentation component 32 may, responsive to an elevated threshold level of exacerbation, present (i) an alert, (ii) one or more vital signs, physiological parameters, or other information associated with patient 34, (iii) a user interface option to remotely interact or consult with patient 34, or other information.

Electronic storage 14 comprises electronic storage media that electronically stores information. The electronic storage media of electronic storage 14 may comprise one or both of system storage that is provided integrally (i.e., substantially non-removable) with system 10 or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., a computing device 18, processor 12, etc.). In some embodiments, electronic storage 14 may be located in a server together with processor 12, in a server that is part of external resources 16, in computing device 18 associated with caregivers, or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), or other electronically readable storage media. Electronic storage 14 may store software algorithms, information determined by processor 12, information received via computing device 18 or other external computing systems, information received from external resources 16, or other information that enables system 10 to function as described herein. By way of a non-limiting example, electronic storage 14 may store the total score for the risk of readmission associated with patient 34 determined by risk determination component 28 of processor 12.

External resources 16 include sources of information (e.g., databases, websites, etc.), external entities participating with system 10 (e.g., a medical records system of a health care provider that stores patient demographics information, patient disease information, facility information, and discharge date information), external home monitoring systems, one or more servers outside of system 10, a network (e.g., the internet), electronic storage, equipment related to Wi-Fi technology, equipment related to Bluetooth® technology, data entry devices, sensors, scanners, or other resources. In some implementations, some or all of the functionality attributed herein to external resources 16 may be provided by resources included in system 10. External resources 16 may be configured to communicate with processor 12, computing device 18, electronic storage 14, or other components of system 10 via wired or wireless connections, via a network (e.g., a local area network or the internet), via cellular technology, via Wi-Fi technology, or via other resources. In some embodiments, external resources 16 includes a telehealth software platform (e.g., Philips eCare Coordinator) that enables one or more care givers to remotely monitor patient 34's vital signs and send one or more surveys to patient 34 about his/her health status. In some embodiments, the telehealth software platform may create new care plans based on patient-specific needs including condition, language, cognition, or other factors.

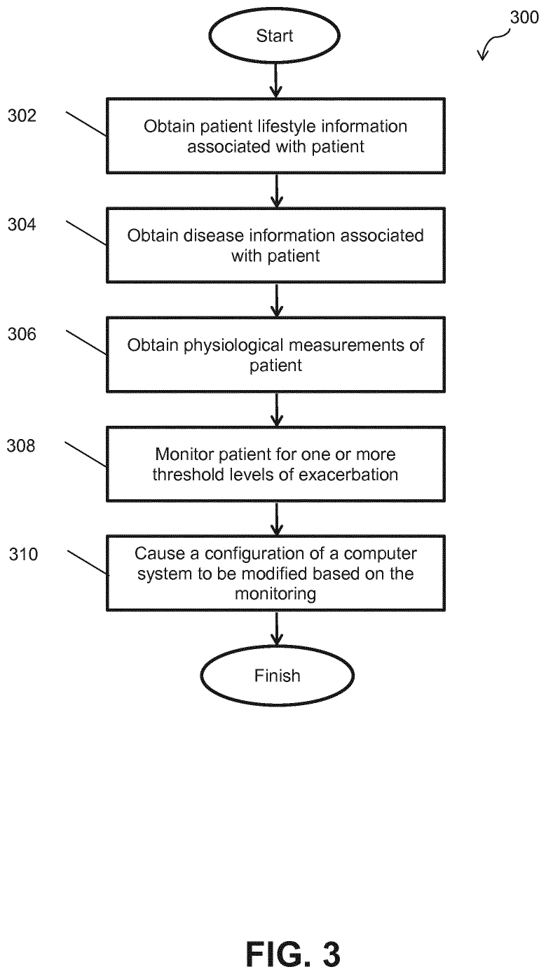

FIG. 3 illustrates a method 300 for facilitating configuration modifications for a patient interface computer system based on a patient-specific risk alert model. Method 300 may be performed with a system. The system comprises one or more processors, or other components. The processors are configured by machine readable instructions to execute computer program components. The computer program components include a communications component, a risk determination component, a configuration management component, a presentation component, or other components. The operations of method 300 presented below are intended to be illustrative. In some embodiments, method 300 may be accomplished with one or more additional operations not described, or without one or more of the operations discussed. Additionally, the order in which the operations of method 300 are illustrated in FIG. 3 and described below is not intended to be limiting.

In some embodiments, method 300 may be implemented in one or more processing devices (e.g., a digital processor, an analog processor, a digital circuit designed to process information, an analog circuit designed to process information, a state machine, or other mechanisms for electronically processing information). The devices may include one or more devices executing some or all of the operations of method 300 in response to instructions stored electronically on an electronic storage medium. The processing devices may include one or more devices configured through hardware, firmware, or software to be specifically designed for execution of one or more of the operations of method 300.

At an operation 302, lifestyle information associated with a patient is obtained. In some embodiments, operation 302 is performed by a processor component the same as or similar to communications component 26 (shown in FIG. 1 and described herein).

At an operation 304, disease information associated with the patient is obtained. In some embodiments, operation 304 is performed by a processor component the same as or similar to communications component 26 (shown in FIG. 1 and described herein).

At an operation 306, one or more physiological measurements of the patient are obtained via one or more sensors. In some embodiments, the sensors are configured to provide real-time signals conveying information indicating the physiological measurements of the patient. In some embodiments, operation 306 is performed by a processor component the same as or similar to communications component 26 (shown in FIG. 1 and described herein).

At an operation 308, the patient is monitored for one or more threshold levels of exacerbation based on a risk alert model, the lifestyle information, the disease information, and the physiological measurements. In some embodiments, operation 308 is performed by a processor component the same as or similar to risk determination component 28 (shown in FIG. 1 and described herein).

At an operation 310, a configuration of the patient interface computer system is caused to be modified based on the monitoring for the threshold levels of exacerbation. In some embodiments, operation 310 is performed by a processor component the same as or similar to configuration management component 30 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A system configured to facilitate configuration modifications for a patient interface computer system based on a patient-specific risk alert model, the system comprising:
an automated medication dispenser configured to dispense a prescribed dosage of medication, and
one or more processors configured by machine-readable instructions to:
obtain lifestyle information associated with a patient;
obtain disease information associated with the patient;
obtain one or more physiological measurements of the patient with one or more sensors, the one or more sensors being configured to provide real-time signals conveying information indicating the one or more physiological measurements of the patient;
monitor the patient for one or more threshold levels of exacerbation based on a risk alert model, the lifestyle information, the disease information, and the one or more physiological measurements; and
cause a configuration of the patient interface computer system to be modified based on the monitoring for the one or more threshold levels of exacerbation,
wherein the causing the configuration of the patient interface computer system to be modified includes changing the dosage of medication dispensed by the automated medication dispenser in response to the patient reaching one or more threshold levels of exacerbation.

2. The system of claim 1, wherein the lifestyle information includes one or more of an age, a gender, social-support, activation, frailty, or severity, the social-support being a parameter associated with a level of support from one or more informal care givers associated with the patient, the activation being a parameter associated with a score value indicating a level of patient engagement to a treatment, the frailty being a parameter associated with a score indicating frailty, and the severity being a parameter associated with a level of disease severity, and wherein the disease information includes one or more of an etiology, a diagnosis, a co-morbid, or a post-discharge, the diagnosis being a parameter associated with systolic and/or diastolic blood pressure, the co-morbid being a parameter associated with one or more co-morbidities of the patient, and the post-discharge being a parameter associated with one or more patient conditions at discharge.

3. The system of claim 1, wherein the one or more processors are configured such that monitoring the patient for the one or more threshold levels of exacerbation comprises (i) determining a baseline probability based on the lifestyle information and the disease information and (ii) determining a probability of exacerbation with the risk alert model based on the determined baseline probability and the one or more physiological measurements.

4. The system of claim 3, wherein the one or more processors are further configured to select a telehealth program for the patient based on the determined baseline probability.

5. The system of claim 3, wherein the baseline probability indicates a probability of exacerbation for a first time period and wherein the risk alert model indicates a probability of exacerbation for a second time period, the first time period being greater than the second time period.

6. The system of claim 1, wherein causing a configuration of the patient interface computer system to be modified includes changing an amount of monitoring of the patient with the one or more sensors.

7. The system of claim 1, wherein the automated medication dispenser comprises a button, and wherein the monitoring the patient includes transmitting via the medication dispenser a report to a care giver a missed medication dose responsive to the button not being pressure within a predetermined amount of time.

8. A method for facilitating configuration modifications for a patient interface computer system based on a patient-specific risk alert model with a system, the system comprising an automated medication dispenser configured to dispense a prescribed dosage of medication and one or more processors, the method comprising:
obtaining, with the one or more processors, lifestyle information associated with a patient;
obtaining, with the one or more processors, disease information associated with the patient;
obtaining, with one or more sensors, one or more physiological measurements of the patient, the one or more sensors being configured to provide real-time signals conveying information indicating the one or more physiological measurements of the patient;
monitoring, with the one or more processors, the patient for one or more threshold levels of exacerbation based on a risk alert model, the lifestyle information, the disease information, and the one or more physiological measurements; and
causing, with the one or more processors, a configuration of the patient interface computer system to be modified based on the monitoring for the one or more threshold levels of exacerbation,
wherein the causing the configuration of the patient interface computer system to be modified includes changing the dosage of medication dispensed by the automated medication dispenser in response to the patient reaching one or more threshold levels of exacerbation.

9. The method of claim 8, wherein the lifestyle information includes one or more of an age, a gender, social-support, activation, frailty, or severity, the social-support being a parameter associated with a level of support from one or more informal care givers associated with the patient, the activation being a parameter associated with a score value indicating a level of patient engagement to a treatment, the frailty being a parameter associated with a score indicating frailty, and the severity being a parameter associated with a level of disease severity, and wherein the disease information includes one or more of an etiology, a diagnosis, a co-morbid, or a post-discharge, the diagnosis being a parameter associated with systolic and/or diastolic blood pressure, the co-morbid being a parameter associated with one or more co-morbidities of the patient, and the post-discharge being a parameter associated with one or more patient conditions at discharge.

10. The method of claim 8, wherein monitoring the patient for the one or more threshold levels of exacerbation comprises (i) determining a baseline probability based on the lifestyle information and the disease information and (ii) determining a probability of exacerbation with the risk alert model based on the determined baseline probability and the one or more physiological measurements.

11. The method of claim 10, further comprising selecting, with the one or more processors, a telehealth program for the patient based on the determined baseline probability.

12. The method of claim 10, wherein the baseline probability indicates a probability of exacerbation for a first time period and wherein the risk alert model indicates a probability of exacerbation for a second time period, the first time period being greater than the second time period.

13. The method of claim 8, wherein causing a configuration of the patient interface computer system to be modified includes changing an amount of monitoring of the patient with the one or more sensors.

14. The method of claim 8, wherein the automated medication dispenser comprises a button, and wherein the monitoring the patient includes transmitting via the medication dispenser a report to a care giver a missed medication dose responsive to the button not being pressure within a predetermined amount of time.

15. A system configured to facilitate configuration modifications for a patient interface computer system based on a patient-specific risk alert model, the system comprising:
means for dispensing a prescribed dosage of medication;
means for obtaining lifestyle information associated with a patient;
means for obtaining disease information associated with the patient;
means for obtaining one or more physiological measurements of the patient, the means for obtaining one or more physiological measurements being configured to provide real-time signals conveying information indicating the one or more physiological measurements of the patient;
means for monitoring the patient for one or more threshold levels of exacerbation based on a risk alert model, the lifestyle information, the disease information, and the one or more physiological measurements; and
means for causing a configuration of the patient interface computer system to be modified based on the monitoring for the one or more threshold levels of exacerbation,
wherein the causing a configuration of the patient interface computer system to be modified includes changing the dosage of medication dispensed by the means for dispensing in response to the patient reaching one or more threshold levels of exacerbation.

16. The system of claim 15, wherein the lifestyle information includes one or more of an age, a gender, social-support, activation, frailty, or severity, the social-support being a parameter associated with a level of support from one or more informal care givers associated with the patient, the activation being a parameter associated with a score value indicating a level of patient engagement to a treatment, the frailty being a parameter associated with a score indicating frailty, and the severity being a parameter associated with a level of disease severity, and wherein the disease information includes one or more of an etiology, a diagnosis, a co-morbid, or a post-discharge, the diagnosis being a parameter associated with systolic and/or diastolic blood pressure, the co-morbid being a parameter associated with one or more co-morbidities of the patient, and the post-discharge being a parameter associated with one or more patient conditions at discharge.

17. The system of claim 15, wherein the means for monitoring the patient for the one or more threshold levels of exacerbation comprises (i) means for determining a baseline probability based on the lifestyle information and the disease information and (ii) means for determining a probability of exacerbation with the risk alert model based on the determined baseline probability and the one or more physiological measurements.

18. The system of claim 17, further comprising means for selecting a telehealth program for the patient based on the determined baseline probability.

19. The system of claim 15, wherein causing a configuration of the patient interface computer system to be modified includes changing an amount of monitoring of the patient with the means for obtaining one or more physiological measurements.

20. The system of claim 15, wherein the automated medication dispenser comprises a button, and wherein the monitoring the patient includes transmitting via the medication dispenser a report to a care giver a missed medication dose responsive to the button not being pressure within a predetermined amount of time.
